(19) United States
(12) Patent Application Publication
(10) Pub. No.: US 2023/0052902 A1
(43) Pub. Date: Feb. 16, 2023

(54) CONDITION VISUALIZATION SYSTEM, SPATIAL CONTROL SYSTEM, CONDITION VISUALIZATION METHOD, AND PROGRAM

(71) Applicant: Panasonic Intellectual Property Management Co., Ltd., Osaka (JP)

(72) Inventors: Yuki WAKI, Osaka (JP); Keita YOSHIMURA, Osaka (JP)

(21) Appl. No.: 17/792,063

(22) PCT Filed: Jan. 15, 2021
(86) PCT No.: PCT/JP2021/001328

§ 371 Date: Jul. 11, 2022

(30) Foreign Application Priority Data
Jan. 31, 2020 (JP) 2020-015863

(51) Int. Cl. A61B 5/00; G16H 40/67; G16H 50/30; A61B 5/16

(52) U.S. Cl. CPC A61B 5/742; G16H 40/67; G16H 50/30; A61B 5/16

(57) ABSTRACT

A condition visualization system includes a first acquisition unit, an estimation unit, a processing unit, an output unit, a second acquisition unit, and a correction information generation unit. The first acquisition unit acquires a user's biometric information. The estimation unit obtains, based on the biometric information, an estimated value representing the user's mental and physical condition. The processing unit generates, based on the estimated value, mental and physical condition information. The output unit outputs the mental and physical condition information to a display unit. The second acquisition unit acquires a subjective value with respect to the user's mental and physical condition. The correction information generation unit generates correction information by using a plurality of data sets, each including the estimated value and the subjective value. The processing unit generates the mental and physical condition information by making correction to the estimated value based on the correction information.

### TECHNICAL FIELD

The present disclosure generally relates to a condition visualization system, a spatial control system, a condition visualization method, and a program. More particularly, the present disclosure relates to a condition visualization system, a spatial control system, a condition visualization method, and a program, all of which are configured or designed to visualize the user's mental and physical condition.

### BACKGROUND ART

A system for estimating the user's mental and physical condition has been known in the art (see, for example, Patent Literature 1).

According to Patent Literature 1, the distribution of autonomic activity indices (to mental and physical condition) that have been collected beforehand according to genders and age groups is stored in advance. In addition, according to Patent Literature 1, the users' autonomic activity indices are estimated based on the distribution of autonomic activity indices according to their genders and age groups.

This increases the accuracy of estimation compared to using a distribution that is not classified according to their genders and age groups.

Nevertheless, even if the distribution of autonomic activity indices of people who have the same gender, and belong to the same age group, as a given user, is used, there still are individual differences between the user and those people of the same gender and the same age group. That is why the result of estimation is not always adequate.

### CITATION LIST

### Patent Literature

Patent Literature 1: JP 2014-140587 A

### SUMMARY OF INVENTION

In view of the foregoing background, it is therefore an object of the present disclosure to provide a condition visualization system, a spatial control system, a condition visualization method, and a program, all of which are configured or designed to notify the user of a result of more accurate estimation of his or her own mental and physical condition.

A condition visualization system according to an aspect of the present disclosure includes a first acquisition unit, an estimation unit, a processing unit, an output unit, a second acquisition unit, and a correction information generation unit. The first acquisition unit acquires a user's biometric information from a measuring unit that measures the biometric information. The estimation unit obtains, based on the biometric information, an estimated value representing the user's mental and physical condition. The processing unit generates, based on the estimated value, mental and physical condition information about the user's mental and physical condition. The output unit outputs the mental and physical condition information to a display unit that conducts a display based on the mental and physical condition information. The second acquisition unit acquires a subjective value that reflects the user's subjective with respect to the user's own mental and physical condition when the estimated value is obtained. The correction information generation unit generates, using a plurality of data sets, each including the estimated value and the subjective value, correction information about correction to be made to the estimated value when the mental and physical condition information is generated. The processing unit generates the mental and physical condition information by making correction to the estimated value based on the correction information.

A spatial control system according to another aspect of the present disclosure includes a spatial controller that performs spatial control based on the mental and physical condition information generated by the condition visualization system described above.

A condition visualization method according to still another aspect of the present disclosure includes a first acquisition step, an estimation step, a processing step, an output step, a second acquisition step, and a correction information generation step. The first acquisition step includes acquiring a user's biometric information from a measuring unit that measures the biometric information. The estimation step includes obtaining, based on the biometric information, an estimated value representing the user's mental and physical condition. The processing step includes generating, based on the estimated value, mental and physical condition information about the user's mental and physical condition. The output step includes outputting the mental and physical condition information to a display unit that conducts a display based on the mental and physical condition information. The second acquisition step includes acquiring a subjective value that reflects the user's subjective with respect to the user's own mental and physical condition when the estimated value is obtained. The correction information generation step includes generating, using a plurality of data sets, each including the estimated value and the subjective value, correction information about correction to be made to the estimated value when the mental and physical condition information is generated. The processing step includes generating the mental and physical condition information by making correction to the estimated value based on the correction information.

A program according to yet another aspect of the present disclosure is designed to cause a computer to perform the condition visualization method described above.

### BRIEF DESCRIPTION OF DRAWINGS

FIG. 1 illustrates a configuration for a condition visualizer as a condition visualization system according to an exemplary embodiment;

FIG. 2 illustrates an exemplary mode of use of the condition visualizer;

FIG. 3 shows how the condition visualizer may operate when updating a corrected estimated value;

FIG. 4 shows how the condition visualizer and a spatial controller according to the exemplary embodiment may operate when generating mental and physical condition information;

FIG. 5A illustrates an exemplary on-screen image in a situation where a result of estimation of a mental and physical condition is displayed; and

FIG. 5B illustrates an exemplary on-screen image in a situation where a subjective value entered is accepted.

### DESCRIPTION OF EMBODIMENTS

Note that the embodiment and its variations to be described below are only an exemplary one of various embodiments and its variations of the present disclosure and should not be construed as limiting. Rather, the exemplary embodiment and variations may be readily modified in various manners depending on a design choice or any other factor without departing from a true spirit and scope of the present disclosure.

### Embodiment

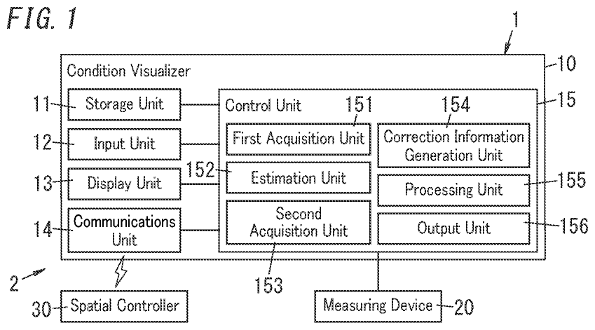

A condition visualization system 1, spatial control system 2, and condition visualization method according to an exemplary embodiment will be described with reference to FIGS. 1-5B.

(1) Overview

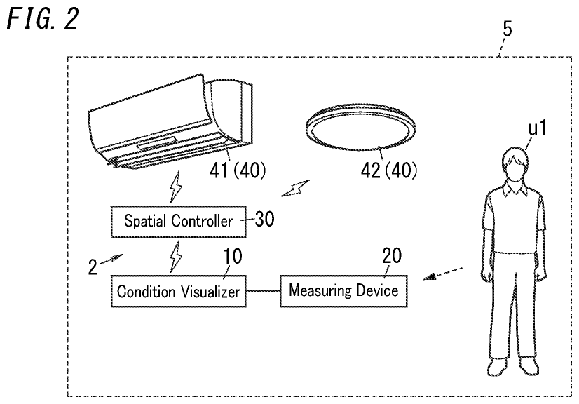

A condition visualizer 10 as an exemplary condition visualization system 1 according to this embodiment is configured to be ready to communicate with a measuring device 20 (measuring unit). A spatial control system 2 according to this embodiment includes the condition visualizer 10 and a spatial controller 30 (see FIGS. 1 and 2).

The measuring device 20 is a device for measuring information about the biometric data (i.e., biometric information) of a user u1 (see FIG. 2) who is present in a space 5 (see FIG. 2). The measuring device 20 may be, for example, a wearable terminal. In this case, examples of the biometric information include an electrocardiogram, a blood pressure, a vascular caliber, a respiratory rate, a pupil diameter, a blood glucose level, a facial expression, an electroencephalogram, a blood flow, and perspiration. In this embodiment, the measuring device 20 may measure, as the biometric information, the user's u1 pulse wave at a predetermined timing for a predetermined period (of one minute, for example). Optionally, the biometric information may be measured on a regular basis (i.e., at regular time intervals (e.g., every minute)).

The condition visualizer 10 acquires the result of measurement (biometric information) made by the measuring device 20 and estimates, based on the result of measurement thus acquired, the user's u1 mental and physical condition. The condition visualizer 10 generates information (mental and physical condition information) based on the result of estimation and outputs the information to a display unit (such as a display unit 13) that conducts a display. The condition visualizer 10 further acquires, from the user u1, a subjective value that reflects the user's u1 subjective with respect to his or her own mental and physical condition. The condition visualizer 10 generates, using a plurality of data sets, each including the result of estimation and the subjective value, correction information about correction to be made to the estimated value when the mental and physical condition information is generated.

If the correction information has been generated, the condition visualizer 10 corrects, using the correction information, the result of estimation, thereby generating mental and physical condition information.

In this embodiment, the condition visualizer 10 estimates, as the mental and physical condition, both an arousal level and a valence level. Each of the arousal level and valence level estimated is expressed within the range from −5 to 5.

The condition visualizer 10 is configured to be ready to communicate with the spatial controller 30 for performing spatial control. As used herein, the “spatial control” refers to controlling the spatial environment by controlling at least one device 40 out of a plurality of devices 40 provided in the space 5. In this embodiment, an air conditioner 41 and a lighting fixture 42 are provided as the plurality of devices 40 in the space 5 as shown in FIG. 2.

The spatial controller 30 controls, in accordance with the mental and physical condition information generated by the condition visualizer 10, at least one device 40 out of the plurality of devices 40 (such as the air conditioner 41 and the lighting fixture 42) provided in the space 5.

For example, if the user u1 is going to take a nap in the space 5, the condition visualizer 10 generates mental and physical condition information of the user u1 who is yet to take a nap. The spatial controller 30 controls at least one device out of the plurality of devices 40 (including the air conditioner 41 and the lighting fixture 42) to allow the user u1 to have a nap in the best condition. In addition, the condition visualizer 10 also generates and stores the mental and physical condition information of the user u1 who has taken a nap.

(2) Configuration

(2.1) Measuring Device

Next, a configuration for the measuring device 20 will be described.

The measuring device 20 includes a computer system including a processor and a memory, for example. The computer system performs the functions of the measuring device 20 by making the processor execute a program stored in the memory. In this embodiment, the program to be executed by the processor is stored in advance in the memory of the computer system. Alternatively, the program may also be distributed after having been stored in a non-transitory storage medium such as a memory card or downloaded via a telecommunications line such as the Internet.

The measuring device 20 measures, as the biometric data of the user u1, the user's u1 pulse wave at a predetermined timing for a predetermined period. The measuring device 20 may be implemented as, for example, a sphygmograph in the shape of a wrist band and configured to measure the user's u1 pulse wave when worn by the user u1 on the wrist.

The measuring device 20 outputs, as the biometric information, the user's u1 pulse wave thus measured (i.e., the result of measurement) to the condition visualizer 10. The measuring device 20 may output the biometric information to the condition visualizer 10 by establishing wireless communication or wired communication with the condition visualizer 10, whichever is appropriate.

Optionally, the measuring device 20 may be configured to measure vibrations on the body surface using a microwave Doppler sensor.

Alternatively, the measuring device 20 may also be configured to measure biometric information other than the pulse wave. The measuring device 20 may be implemented as, for example, a head-mounted electroencephalograph for measuring the user's u1 electroencephalogram when worn by the user u1 on the head. Still alternatively, the measuring device 20 may also be implemented as a camera device to measure the user's u1 pupil diameter or facial expression, for example. Yet alternatively, the measuring device 20 may also be implemented as a microphone device to measure the user's u1 voice and breath sound, for example. Optionally, the spatial control system 2 may include a plurality of measuring devices 20 and configured to measure multiple types of biometric information of the user's u1.

(2.2) Condition Visualizer

Next, a configuration for the condition visualizer 10 will be described with reference to FIG. 1.

As shown in FIG. 1, the condition visualizer 10 includes a storage unit 11, an input unit 12, a display unit 13, a communications unit 14, and a control unit 15.

The condition visualizer 10 includes a computer system including a processor and a memory, for example. The computer system performs the functions of the control unit 15 by making the processor execute a program stored in the memory. In this embodiment, the program to be executed by the processor is stored in advance in the memory of the computer system. Alternatively, the program may also be distributed after having been stored in a non-transitory storage medium such as a memory card or downloaded via a telecommunications line such as the Internet.

The storage unit 11 is implemented as a readable and writable memory. The storage unit 11 may be a flash memory, for example. The storage unit 11 stores, in association with each other, an estimated value, which is a value calculated based on the biometric information with respect to each individual user u1 to represent the user's u1 mental and physical condition, and a subjective value acquired from the user u1. In addition, the storage unit 11 further stores, in association with each other, the estimated value and an estimated value that has been corrected (hereinafter referred to as a “corrected estimated value”) with respect to each individual user u1. Specifically, the storage unit 11 stores an estimate table shown in the following Table 1. In this embodiment, the storage unit 11 stores an estimate table about the arousal level and an estimate table about the valence level with respect to each individual user u1.

TABLE 1

| | | Corrected |
|---|---|---|
| Estimated | Subjective | estimated |
| value | value | value |
| 2 | 2.2 | 2.2 |
| 2 | 2.4 | |
| 2 | 2.0 | |
| 2 | 0.5 | |
| 2.1 | 2.2 | 2.2 |
| 2.1 | 1.8 | |
| 2.2 | 2.3 | 2.4 |
| 2.2 | 2.4 | |
| 2.2 | 2.5 | |

Examples of the input unit 12 include a keyboard, a mouse, and a touchscreen panel. The input unit 12 accepts input of a subjective value that reflects subjective with respect to the mental and physical condition. In this embodiment, the input unit 12 accepts input of a subjective value with respect to the arousal level and a subjective value with respect to the valence level.

The display unit 13 is a thin display device such as a liquid crystal display or an organic electroluminescent (EL) display. The display unit 13 displays an on-screen image about the mental and physical condition information. The display unit 13 displays mental and physical condition information with respect to the arousal level (as first mental and physical condition information) and mental and physical condition information with respect to the valence level (as second mental and physical condition information). The display unit 13 may display, for example, a combination of the first mental and physical condition information and the second mental and physical condition information as coordinates on a two-dimensional matrix.

In addition, the display unit 13 further displays the subjective value with respect to the arousal level and the subjective value with respect to the valence level that have been entered via the input unit 12. For example, the display unit 13 may display a combination of the subjective value with respect to the arousal level and the subjective value with respect to the valence level as coordinates on a two-dimensional matrix.

The communications unit 14 includes a communications interface for communicating with the spatial controller 30. The communications interface is designed to communicate with, for example, the spatial controller 30 either wirelessly or via cables.

As shown in FIG. 1, the control unit 15 includes a first acquisition unit 151, an estimation unit 152, a second acquisition unit 153, a correction information generation unit 154, a processing unit 155, and an output unit 156.

The first acquisition unit 151 acquires the user's u1 biometric information from the measuring device 20.

The estimation unit 152 obtains, based on the biometric information, an estimated value representing the user's u1 mental and physical condition. The estimation unit 152 obtains, as a plurality of estimated values, a first-type estimated value with respect to a first type of condition representing the mental and physical condition and a second-type estimated value with respect to a second type of condition representing the mental and physical condition. In this embodiment, the first type corresponds to the arousal level and the second type corresponds to the valence level.

The estimation unit 152 calculates the first-type estimated value by using the pulse wave. For example, the estimation unit 152 divides a range in which a person's pulse wave normally falls into a plurality of subranges and associates each of these subranges divided with any of the values of −5 to 5. The estimation unit 152 calculates, as the first-type estimated value, a value associated with the biometric information acquired by the first acquisition unit 151.

The estimation unit 152 calculates the second-type estimated value by using the pulse wave. For example, the estimation unit 152 calculates an “LF/HF” value based on HF (Hi Frequency) and LF (Low Frequency) of a power spectrum in the frequency range of the pulse wave variation. The estimation unit 152 transforms the “LF/HF” value into a natural logarithm and calculates the second-type estimated value based on the transformed value. Alternatively, the estimation unit 152 may also use, as an index for calculating the second-type estimated value, an index different from the “LF/HF” value. For example, the estimation unit 152 may also use, as an index for calculating the second-type estimated value, a root mean square of successive difference (RMSSD), which is an index to the activation of a parasympathetic nervous system, or a coefficient of variation in electrocardiographic R-R intervals (CVRR).

The second acquisition unit 153 acquires a subjective value that reflects the user's u1 subjective with respect to his or her own mental and physical condition when the estimated value is obtained. In this embodiment, the second acquisition unit 153 acquires the subjective value that has been accepted by the input unit 12.

The second acquisition unit 153 acquires, while the display unit 13 is displaying the first mental and physical condition information and the second mental and physical condition information, coordinates specified on the two-dimensional matrix. The second acquisition unit 153 also acquires one value, corresponding to the first type (arousal level), out of two values represented by the coordinates thus acquired, as a subjective value associated with the first-type estimated value, and further acquires the other value, corresponding to the second type (valence level), out of the two values, as a subjective value associated with to the second-type estimated value.

On acquiring the subjective value, the second acquisition unit 153 stores, in the storage unit 11, the subjective value and an estimated value representing the mental and physical condition information and obtained by the estimation unit 152, in association with each other. Specifically, the second acquisition unit 153 stores, in association with each other, a subjective value corresponding to the arousal level and the first-type estimated value in the estimate table with respect to the arousal level in the storage unit 11. In addition, the second acquisition unit 153 also stores, in association with each other, a subjective value corresponding to the valence level and the second-type estimated value in the estimate table with respect to the arousal level in the storage unit 11.

The correction information generation unit 154 generates, using a plurality of data sets, each including the estimated value and the subjective value, correction information about correction to be made to the estimated value when the mental and physical condition information is generated. In this case, the correction information includes the corrected estimated value described above.

In this embodiment, the correction information generation unit 154 generates (i.e., updates) the correction information when an update condition for updating the correction information is satisfied. In this case, the update condition is that the current date and time be the predetermined date and time. For example, when it is 9:00 am on the first day of each month, the correction information generation unit 154 generates (updates) the correction information. In this manner, a corrected estimated value of the correction information is generated (updated) at regular intervals.

The correction information generation unit 154 calculates, using at least a predetermined number of (e.g., three) data sets, each having the same estimated value, out of the plurality of data sets, an average value of a plurality of subjective values included in the at least the predetermined number of data sets as the corrected estimated value. The correction information generation unit 154 generates, as correction information with respect to the estimated value, the corrected estimated value thus calculated. In this case, the correction information generation unit 154 calculates, when finding the difference between the estimated value and the subjective value equal to or greater than a predetermined value (e.g., a value of “1”) in the plurality of data sets each having the same estimated value, the average value with that subjective value excluded (which will be hereinafter referred to as a “subjective value to be excluded”). At this time, the correction information generation unit 154 calculates the average value when finding the number of the other data sets, except the data set including the excluded subjective value, equal to or greater than a predetermined value. For example, as shown in Table 1, the difference (i.e., a value of “1.5”) between the subjective value of “0.5” associated with the estimated value of “2.0” and the estimated value of “2.0” is equal to or greater than a predetermined value (e.g., a value of “1”). Thus, the correction information generation unit 154 calculates a corrected estimated value with respect to the estimated value of “2.0” with the subjective value of “0.5” excluded. Note that when finding the number of the other data sets, except the data set including the excluded subjective value, equal to or greater than a predetermined number, the correction information generation unit 154 calculates the average value by the processing to be described later.

The correction information generation unit 154 calculates, when there are less than the predetermined number of the data sets, each including a particular estimated value, an average value as a corrected estimated value using subjective values included in all of a plurality of data sets including the particular estimated value and subjective values included in all of another plurality of the data sets including another estimated value continuous with the particular estimated value. The correction information generation unit 154 generates the corrected estimated value thus calculated as the correction information with respect to the estimated value. In this case, the correction information generation unit 154 also calculates, when finding the difference between the estimated value and the subjective value equal to or greater than a predetermined value (e.g., a value of “1”) in the plurality of data sets each having the same estimated value, the average value with that subjective value excluded. For example, as shown in Table 1, the number of the subjective values associated with an estimated value of “2.1” is less than the predetermined number. Thus, the correction information generation unit 154 uses two estimated values of “2.0” and “2.2,” which are continuous with, and different from, the estimated value of “2.1,” to generate the correction information. Specifically, the correction information generation unit 154 calculates the average value of the subjective values (e.g., a value of “2.2” in this example) using all subjective values associated with the estimated value of “2.1,” all subjective values associated with the estimated value of “2.0,” and all subjective values associated with the estimated value of “2.2.”

In this embodiment, the correction information generation unit 154 is configured to calculate the average value of the subjective values as a corrected estimated value. However, this configuration is only an example and should not be construed as limiting. Alternatively, the correction information generation unit 154 may also calculate a median of the subjective values as the corrected estimated value.

Also, in the embodiment described above, the correction information generation unit 154 is configured to, when there are less than the predetermined number of data sets, each including a particular estimated value, calculate the corrected estimated value by using all data sets of both the two values that are continuous with, and precede and follow, the particular estimated value. However, this configuration is only an example and should not be construed as limiting. Alternatively, the correction information generation unit 154 may also calculate, when there are less than the predetermined number of data sets, each including a particular estimated value, the corrected estimated value by using all data sets, each including the particular estimated value, and all data sets, each including only one of the preceding and following values continuous with the particular estimated value. That is to say, the correction information generation unit 154 calculates, when there are less than the predetermined number of data sets, each including a particular estimated value, the corrected estimated value by using all data sets, each including the particular estimated value, and all data sets, each including at least one of the preceding and following values that are continuous with the particular estimated value.

The processing unit 155 generates, based on the estimated value, mental and physical condition information about the user's u1 mental and physical condition. The processing unit 155 generates the mental and physical condition information by making correction to the estimated value based on the correction information. More specifically, the processing unit 155 generates the mental and physical condition information by making, if there is a corrected estimated value associated with an estimated value, correction to the estimated value using the corrected estimated value.

The processing unit 155 generates, based on the first-type estimated value, first mental and physical condition information as a piece of the mental and physical condition information and also generates, based on the second-type estimated value, second mental and physical condition information as another piece of the mental and physical condition information. The processing unit 155 generates, if there is a corrected estimated value associated with the estimated value (i.e., the first-type estimated value) calculated by the estimation unit 152, first mental and physical condition information including the corrected estimated value. The processing unit 155 generates, if there is a corrected estimated value associated with the estimated value (i.e., the second-type estimated value) calculated by the estimation unit 152, second mental and physical condition information including the corrected estimated value.

The processing unit 155 generates, if there are no corrected estimated values associated with the estimated value (i.e., the first-type estimated value) calculated by the estimation unit 152, first mental and physical condition information including the first-type estimated value. The processing unit 155 generates, if there are no corrected estimated values associated with the estimated value (i.e., the second-type estimated value) calculated by the estimation unit 152, second mental and physical condition information including the second-type estimated value.

The output unit 156 outputs the mental and physical condition information to the display unit 13 that conducts a display in accordance with the mental and physical condition information. Specifically, the output unit 156 outputs the first mental and physical condition information and the second mental and physical condition information to the display unit 13 to have a combination of the first mental and physical condition information and the second mental and physical condition information displayed as coordinates on a two-dimensional matrix.

(2.3) Spatial Controller

The spatial controller 30 includes a computer system including a processor and a memory for example. The computer system performs the functions of the spatial controller 30 by making the processor execute a program stored in the memory. In this embodiment, the program to be executed by the processor is stored in advance in the memory of the computer system.

Alternatively, the program may also be distributed after having been stored in a non-transitory storage medium such as a memory card or downloaded via a telecommunications line such as the Internet.

The spatial controller 30 includes a communications interface for communicating with the condition visualizer 10. The spatial controller 30 also includes a communications interface for communicating with the plurality of devices 40. Optionally, the communications interface for communicating with the condition visualizer 10 and the communications interface for communicating with the plurality of devices 40 may be the same communications interface.

The spatial controller 30 performs spatial control based on the mental and physical condition information generated by the condition visualizer 10. Specifically, the spatial controller 30 controls the operation of at least one device 40 out of the plurality of devices 40 provided in the space 5 in accordance with the first mental and physical condition information and the second mental and physical condition information that have been generated by the condition visualizer 10.

The spatial controller 30 controls, in accordance with the first mental and physical condition information and the second mental and physical condition information that have been generated, at least one of the plurality of devices 40 (including the air conditioner 41 and the lighting fixture 42) to allow the user u1 to take a nap in the best condition. For example, the spatial controller 30 controls at least one of the air volume, air direction, temperature setting, or humidity setting of the air conditioner 41 to allow the user u1 to take a nap in the best condition. In addition, the spatial controller 30 also controls at least one of the illuminance, color temperature, or flickering pattern of the lighting fixture 42 to allow the user u1 to take a nap in the best condition.

Optionally, the spatial controller 30 may also control a sound, odor, or any other parameter, not just controlling the air conditioner 41 or the lighting fixture 42.

(3) Operation

(3.1) Operation when Correction Information is Updated

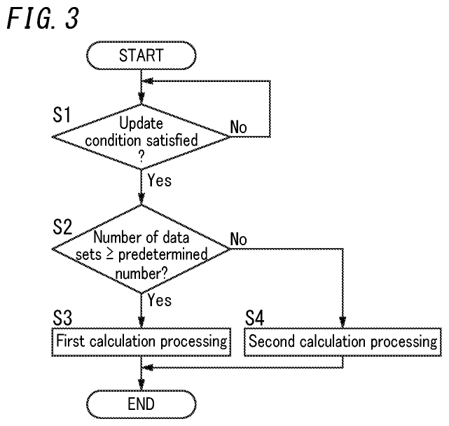

Next, it will be described with reference to FIG. 3 how the condition visualizer 10 operates when the correction information is updated.

The correction information generation unit 154 determines whether an update condition for updating the correction information is satisfied or not (in Step S1). In this example, the correction information generation unit 154 determines whether the current date and time is the predetermined date and time or not.

If a decision is made that the update condition should not be satisfied (if the answer is NO in Step S1), then the process waits for the update condition to be satisfied.

On the other hand, if a decision is made that the update condition be satisfied (if the answer is YES in Step S1), then the correction information generation unit 154 performs the following processing steps on an estimated value basis.

The correction information generation unit 154 determines whether in a plurality of data sets, each including the same estimated value, the number of the other data sets, except a data set including the estimated value to be excluded, is equal to or greater than a predetermined number (in Step S2).

If a decision is made that the number of the other data sets be equal to or greater than the predetermined number (if the answer is YES in Step S2), then the correction information generation unit 154 performs first calculation processing using all of the other data sets (in Step S3). Specifically, the correction information generation unit 154 calculates, as a corrected estimated value, the average value of the subjective values included in the other data sets.

On the other hand, if a decision is made that the number of the other data sets should not be equal to or greater than the predetermined number (i.e., be less than the predetermined number) (if the answer is NO in Step S2), then the correction information generation unit 154 performs second calculation processing (in Step S4). Specifically, the correction information generation unit 154 calculates, as a corrected estimated value, the average value by using the subjective values included in the other data sets and the subjective values included in another plurality of data sets including another estimated value that is a value continuous with the estimated value included in the other data sets.

(3.2) Operation when Mental and Physical Condition Information is Generated

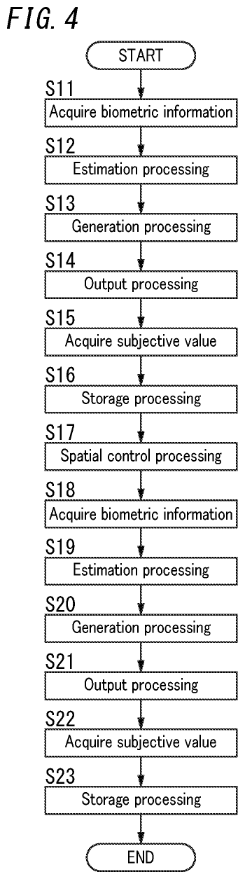

Next, it will be described with reference to FIG. 4 how the condition visualizer 10 and the spatial controller 30 operate when the condition visualizer 10 generates the mental and physical condition information and the spatial controller 30 controls the device 40.

The first acquisition unit 151 of the condition visualizer 10 acquires biometric information (pulse wave) of the user u1 from the measuring device 20 before the user u1 takes a nap (in Step S11).

The estimation unit 152 of the condition visualizer 10 performs estimation processing (in Step S12). The estimation unit 152 obtains, based on the biometric information, an estimated value representing the user's u1 mental and physical condition. Specifically, the estimation unit 152 obtains, using the user's u1 pulse wave acquired in Step S1, a first-type estimated value and a second-type estimated value as respective estimated values.

The processing unit 155 of the condition visualizer 10 performs the processing of generating mental and physical condition information (in Step S13). If the correction information generation unit 154 has generated the correction information, the processing unit 155 corrects the estimated value using the correction information, thereby generating mental and physical condition information. Specifically, the processing unit 155 generates first mental and physical condition information including a corrected estimated value associated with the estimated value (first-type estimated value) calculated in Step S12. The processing unit 155 also generates second mental and physical condition information including a corrected estimated value associated with the estimated value (second-type estimated value) calculated in Step S12. Note that if there is no corrected estimated value associated with the estimated value (first-type estimated value) calculated in Step S12, then the processing unit 155 generates first mental and physical condition information including the first-type estimated value. Likewise, if there is no corrected estimated value associated with the estimated value (second-type estimated value) calculated in Step S12, then the processing unit 155 generates second mental and physical condition information including the second-type estimated value.

The output unit 156 of the condition visualizer 10 performs output processing (in Step S14). The output unit 156 outputs the mental and physical condition information to the display unit 13 that conducts a display based on the mental and physical condition information. Specifically, the output unit 156 outputs the first mental and physical condition information and the second mental and physical condition information to the display unit 13 to have a combination of the first mental and physical condition information and the second mental and physical condition information, which have been generated in Step S13, displayed as coordinates on a two-dimensional matrix.

The second acquisition unit 153 of the condition visualizer 10 acquires subjective values according to the user's u1 subjective when the estimated values are obtained (in Step S15). Specifically, while the display unit 13 is displaying the first mental and physical condition information and the second mental and physical condition information in Step S14, the second acquisition unit 153 acquires coordinates specified on the two-dimensional matrix. The second acquisition unit 153 also acquires one value, corresponding to the first type (arousal level), out of the two values represented by the coordinates thus acquired, as a subjective value associated with the first-type estimated value, and further acquires the other value, corresponding to the second type (valence level), out of the two values, as a subjective value associated with the second-type estimated value.

The second acquisition unit 153 performs storage processing (in Step S16). The second acquisition unit 153 stores, in the storage unit 11, the subjective values acquired in Step S15 and the estimated values obtained by the estimation unit 152 in Step S12 in association with each other. Specifically, the second acquisition unit 153 stores, in association with each other, the subjective value corresponding to the arousal level and acquired in Step S15 and the first-type estimated value obtained in Step S12 in the estimate table with respect to the arousal level in the storage unit 11. In addition, the second acquisition unit 153 also stores, in association with each other, the subjective value corresponding to the valence level and acquired in Step S15 and the second-type estimated value obtained in Step S12 in the estimate table with respect to the arousal level in the storage unit 11.

The spatial controller 30 performs spatial control processing while the user u1 is taking a nap (in Step S17). Specifically, the spatial controller 30 controls the operation of at least one device 40 out of the plurality of devices 40 provided in the space 5 in accordance with the first mental and physical condition information and the second mental and physical condition information that have been generated by the condition visualizer 10.

The first acquisition unit 151 of the condition visualizer 10 acquires the user's u1 biometric information (pulse wave) from the measuring device 20 after the user u1 has taken a nap (in Step S18).

The estimation unit 152 of the condition visualizer 10 performs estimation processing (in Step S19). Specifically, the estimation unit 152 obtains, based on the user's u1 pulse wave acquired in Step S18, a first-type estimated value and a second-type estimated value as respective estimated values.

The processing unit 155 of the condition visualizer 10 performs the processing of generating the mental and physical condition information (in Step S20). Specifically, if the correction information generation unit 154 has generated the correction information, the processing unit 155 generates first mental and physical condition information including a corrected estimated value associated with the first-type estimated value calculated in Step S19. The processing unit 155 also generates second mental and physical condition information including a corrected estimated value associated with the second-type estimated value calculated in Step S19. Note that if there is no corrected estimated value associated with the first-type estimated value calculated in Step S19, then the processing unit 155 generates first mental and physical condition information including the first-type estimated value. Likewise, if there is no corrected estimated value associated with the second-type estimated value calculated in Step S19, then the processing unit 155 generates second mental and physical condition information including the second-type estimated value.

The output unit 156 of the condition visualizer 10 performs output processing (in Step S21). The output unit 156 outputs the mental and physical condition information to the display unit 13 that conducts a display based on the mental and physical condition information. Specifically, the output unit 156 outputs the first mental and physical condition information and the second mental and physical condition information to the display unit 13 to have a combination of the first mental and physical condition information and the second mental and physical condition information, which have been generated in Step S20, displayed as coordinates on a two-dimensional matrix.

The second acquisition unit 153 of the condition visualizer 10 acquires subjective values according to the user's u1 subjective when the estimated values are obtained (in Step S22). Specifically, while the display unit 13 is displaying the first mental and physical condition information and the second mental and physical condition information in Step S21, the second acquisition unit 153 acquires coordinates specified on the two-dimensional matrix. The second acquisition unit 153 also acquires one value, corresponding to the first type (arousal level), out of the two values represented by the coordinates thus acquired, as a subjective value associated with the first-type estimated value, and further acquires the other value, corresponding to the second type (valence level), out of the two values, as a subjective value associated with the second-type estimated value.

The second acquisition unit 153 performs storage processing (in Step S23). The second acquisition unit 153 stores, in the storage unit 11, the subjective values acquired in Step S22 and the estimated values obtained by the estimation unit 152 in Step S19 in association with each other. Specifically, the second acquisition unit 153 stores, in association with each other, the subjective value corresponding to the arousal level and acquired in Step S22 and the first-type estimated value obtained in Step S19 in the estimate table with respect to the arousal level in the storage unit 11. In addition, the second acquisition unit 153 also stores, in association with each other, the subjective value corresponding to the valence level and acquired in Step S22 and the second-type estimated value obtained in Step S19 in the estimate table with respect to the arousal level in the storage unit 11.

(4) Exemplary Display

Next, an exemplary on-screen image G10 will be described on which the display unit 13 displays a combination of the first mental and physical condition information and the second mental and physical condition information as coordinates on a two-dimensional matrix.

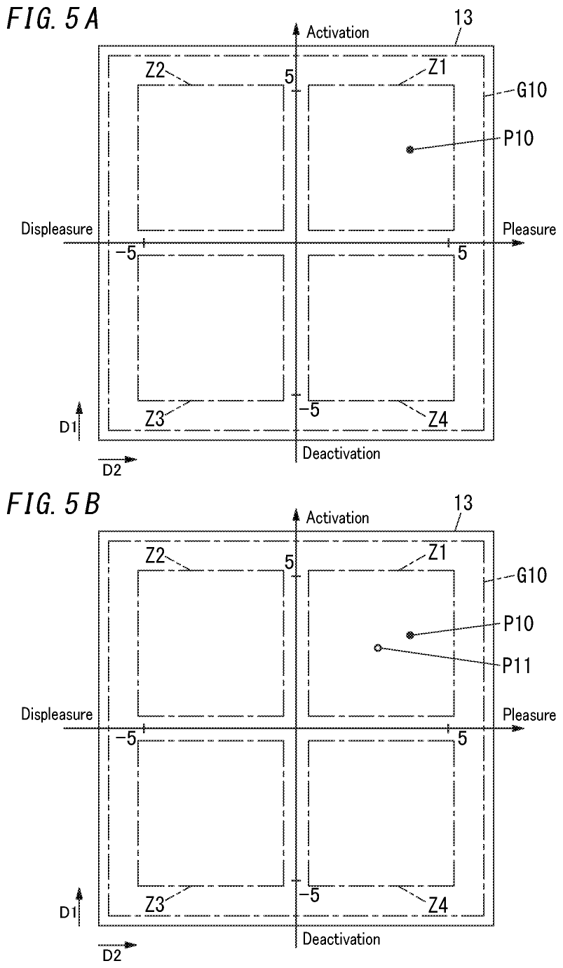

As shown in FIG. 5A, on the on-screen image G10, a first direction D1 defines an axial direction indicating the arousal level and a second direction D2, perpendicular to the first direction D1, defines an axial direction indicating the valence level. The display unit 13 determines, in accordance with the first mental and physical condition information provided by the output unit 156, a coordinate corresponding to the first mental and physical condition information (first value) in the axial direction indicating the arousal level. In addition, the display unit 13 also determines, in accordance with the second mental and physical condition information provided by the output unit 156, a coordinate corresponding to the second mental and physical condition information (second value) in the axial direction indicating the valence level. Then, the display unit 13 determines and displays a point P10, of which the coordinates are the first and second values thus determined (see FIG. 5A).

The input unit 12 accepts, while the display unit 13 is displaying the first mental and physical condition information and the second mental and physical condition information on the on-screen image G10, via the user's u1 input, the coordinates corresponding to the combination of a subjective value with respect to the arousal level and a subjective value with respect to the valence level. The user clicks, by operating a mouse, at a location on the on-screen image G10 corresponding to the combination of the subjective value with respect to the arousal level and the subjective value with respect to the valence level. To show the clicked location to the user, the display unit 13 displays a point P11 at the clicked location (see FIG. 5B).

In addition, the input unit 12 further determines the coordinates of the location where the user has made the click. The second acquisition unit 153 acquires the coordinates (i.e., the coordinates of the point P) determined by the input unit 12. The second acquisition unit 153 acquires, based on the coordinates thus acquired, a subjective value associated with the first-type estimated value and a subjective value associated with the second-type estimated value.

In this embodiment, a two-dimensional psychological model (such as a Russell's circumplex model), which uses the valence level and arousal level as two indices, is used as a model representing the user's u1 mental and physical condition (see FIGS. 5A and 5B). In the two-dimensional psychological model shown in FIGS. 5A and 5B, the axis aligned with the second direction D2 (i.e., the X-axis) indicates the valence level, while the axis aligned with the first direction D1 (i.e., the Y-axis) indicates the arousal level. The valence level in the positive X-axis range indicates “pleasure,” while the valence level in the negative X-axis range indicates “displeasure.” As for the valence level, the degree of pleasure increases as the level (i.e., the absolute value) increases in the positive X-axis range. Meanwhile, the degree of displeasure increases (i.e., the degree of pleasure decreases) as the level (i.e., the absolute value) increases in the negative X-axis range. The arousal level in the positive Y-axis range indicates “activation,” while the arousal level in the negative Y-axis range indicates “deactivation.” As for the arousal level, the degree of activation increases as the level (i.e., the absolute value) increases in the positive Y-axis range. Meanwhile, the degree of deactivation increases (i.e., the degree of activation decreases) as the level (i.e., the absolute value) increases in the negative Y-axis range. The subject's emotions (psychological conditions) have their types classified according to the domain of the two-dimensional model. For example, if the point P10 displayed is present in the first domain Z1, it indicates that the user u1 is in a clear mental and physical condition. If the point P10 displayed is present in the second domain Z2, it indicates that the user u1 is in a stressed mental and physical condition. If the point P10 displayed is present in the third domain Z3, it indicates that the user u1 is in a fatigued mental and physical condition. If the point P10 displayed is present in the fourth domain Z4, it indicates that the user u1 is in a relaxed mental and physical condition.

(5) Advantages

As can be seen from the foregoing description, a condition visualization system 1 (condition visualizer 10) according to this embodiment includes a first acquisition unit 151, an estimation unit 152, a processing unit 155, an output unit 156, a second acquisition unit 153, and a correction information generation unit 154. The first acquisition unit 151 acquires a user's u1 biometric information from a measuring unit (e.g., a measuring device 20) that measures the biometric information. The estimation unit 152 obtains, based on the biometric information, an estimated value representing the user's u1 mental and physical condition. The processing unit 155 generates, based on the estimated value, mental and physical condition information about the user's u1 mental and physical condition. The output unit 156 outputs the mental and physical condition information to a display unit (e.g., display unit 13) that conducts a display based on the mental and physical condition information. The second acquisition unit 153 acquires a subjective value that reflects the user's u1 subjective with respect to his or her own mental and physical condition when the estimated value is obtained. The correction information generation unit 154 generates, using a plurality of data sets, each including the estimated value and the subjective value, correction information about correction to be made to the estimated value when the mental and physical condition information is generated. The processing unit 155 generates the mental and physical condition information by making correction to the estimated value using the correction information when the correction information generation unit 154 generates the correction information.

According to this configuration, the estimated value is corrected based on the correction information that has been generated using the subjective value, thus making the estimated value more approximate to a value that reflects the user's u1 subjective. This enables notifying the user of a result of more accurate estimation of his or her own mental and physical condition.

In addition, according to this embodiment, each estimated value has a stride of 0.1 as shown in Table 1. Meanwhile, the corrected estimated value is the average value of subjective values associated with the estimated values. Thus, although the estimated values have a constant stride, the corrected estimated values may have variable strides.

The corrected estimated value does not have to be the average value of subjective values associated with the estimated values but may also have varied strides. For example, in the range of values that the estimated value may have (e.g., in the range from −5 to 5 in this example), the stride of a corrected estimated value associated with an estimated value close to a median may be smaller than the stride of a corrected estimated value associated with an estimated value close to the maximum or minimum value of the range of values that the estimated value may have.

(6) Variations

Note that the embodiment described above is only an exemplary one of various embodiments of the present disclosure and should not be construed as limiting. Rather, the exemplary embodiment described above may be readily modified in various manners depending on a design choice or any other factor without departing from the scope of the present disclosure.

Next, variations of the exemplary embodiment will be enumerated one after another. Note that the variations to be described below may be adopted in combination as appropriate.

(6.1) First Variation

In the embodiment described above, the correction information generation unit 154 calculates, using at least a predetermined number of subjective values associated with estimated values, the average value of the subjective values as a corrected estimated value. Thus, when the plurality of estimated values includes a first estimated value and a second estimated value, of which values are continuous with each other, the first estimated value may be smaller than the second estimated value, and a corrected estimated value, associated with the first estimated value (hereinafter referred to as a “first corrected estimated value”) may be larger than a corrected estimated value, associated with the second estimated value (hereinafter referred to as a “second corrected estimated value”) in some cases.

Thus, if this is the case, the correction information generation unit 154 may calculate an average value of: a plurality of subjective values associated with a third estimated value that is continuous with, and larger than, the second estimated value; a plurality of subjective values associated with the first estimated value; and a plurality of subjective values associated with the second estimated value to generate the correction information including the average value as the corrected estimated value associated with the second estimated value.

For example, in the following Table 2, the average value of three subjective values associated with an estimated value of “2” is “2.2.” Also, the average value of three subjective values associated with an estimated value of “2.1” is “2.0.” Thus, if the estimated value of “2” is referred to as a “first estimated value” and the estimated value of “2.1” is referred to as a “second estimated value,” then the first estimated value and the second estimated value satisfy the relation described in the second last paragraph. Thus, the correction information generation unit 154 calculates the average value of: a plurality of subjective values associated with a third estimated value (i.e., the value of “2.2” in this example) that is continuous with, and larger than, the second estimated value (i.e., the value of “2.1” in this example); a plurality of subjective values associated with the first estimated value; and a plurality of subjective values associated with the second estimated value.

Specifically, the correction information generation unit 154 calculates an average value of “2.2” of: a plurality of subjective values (i.e., values of “2.3,” “2.4,” and “2.5”) associated with a third estimated value; a plurality of subjective values (i.e., values of “2.2,” “2.4,” and “2.0”) associated with a first estimated value; and a plurality of subjective values (i.e., values of “2.2,” “1.8,” and “2.0”) associated with a second estimated value (see the following Table 2).

TABLE 2

| | | Corrected |
|---|---|---|
| Estimated | Subjective | estimated |
| value | value | value |
| 2 | 2.2 | 2.2 |
| 2 | 2.4 | |
| 2 | 2.0 | |
| 2.1 | 2.2 | 2.2 |
| 2.1 | 1.8 | |
| 2.1 | 2.0 | |
| 2.2 | 2.3 | 2.4 |
| 2.2 | 2.4 | |
| 2.2 | 2.5 | |

(6.2) Second Variation

In the embodiment described above, the association between the estimated values and the subjective values and the association between the estimated values and the corrected estimated values are managed using a single estimate table. However, this configuration is only an example and should not be construed as limiting.

Alternatively, the association between the estimated values and the subjective values and the association between the estimated values and the corrected estimated values may also be managed separately using two different tables.

(6.3) Third Variation

In the embodiment described above, the update condition is supposed to be that the current date and time be the predetermined date and time. However, this is only an exemplary update condition and should not be construed as limiting.

Alternatively, the update condition may also be that a differential value between a corrected estimated value and a subjective value associated with the corrected estimated value be equal to or greater than a predetermined value (predefined value). The correction information generation unit 154 updates a corrected estimated value when finding the differential value between the corrected estimated value and a subjective value associated with the corrected estimated value equal to or greater than the predefined value. More specifically, the correction information generation unit 154 updates a corrected estimated value when finding the differential value between the corrected estimated value and the average value of a plurality of subjective values associated with the corrected estimated value equal to or greater than the predefined value. Alternatively, the correction information generation unit 154 may update a corrected estimated value when finding the differential value between the corrected estimated value and the maximum subjective value out of a plurality of subjective values associated with the corrected estimated value equal to or greater than the predefined value.

Still alternatively, the update condition may also be that the number of subjective values, of which the difference from the estimated value is less than a predetermined value, be equal to or greater than a predetermined number. The correction information generation unit 154 updates a corrected estimated value when finding the number of subjective values, of which the difference from the estimated value is less than a predetermined value, equal to or greater than a predetermined number. In that case, the correction information generation unit 154 does not calculate any corrected estimated value with respect to an estimated value until the number of subjective values, of which the difference from the estimated value is less than a predetermined value, becomes equal to or greater than the predetermined number.

(6.4) Fourth Variation

In the embodiment described above, the storage unit 11 is configured to store the estimated values and the corrected estimated values in association with each other. However, this is only an example and should not be construed as limiting.

Alternatively, the storage unit 11 may store a differential value between an estimated value and a corrected estimated value associated with the estimated value, in association with the estimated value. In that case, the processing unit 155 calculates a corrected estimated value by adding, to an estimated value, a differential value associated with the estimated value. The processing unit 155 generates mental and physical condition information including the corrected estimated value thus calculated.

(6.5) Fifth Variation

In the embodiment described above, the condition visualizer 10 is configured to acquire biometric information before and after the user u1 takes a nap. However, this configuration is only an example and should not be construed as limiting.

Alternatively, the condition visualizer 10 may acquire biometric information while the user u1 is taking a nap (in other words, while the spatial controller 30 is performing spatial control).

In that case, while the user u1 is taking a nap, the condition visualizer 10 cannot acquire any subjective value but may acquire an estimated value. This allows the condition visualizer 10 to estimate a variation in the mental and physical condition of the user u1 who is taking a nap.

(6.6) Sixth Variation

In the embodiment described above, the input range of the subjective values may be limited. For example, with the coordinates of a combination of first mental and physical condition information and second mental and physical condition information (i.e., the point P10 shown in FIGS. 5A and 5B) regarded as a reference point, the input range of subjective values may be defined by the range from —1 to 1 in the first direction D1 and the range from —1 to 1 in the second direction D2. The input unit 12 accepts the input of a subjective value falling within the input range of subjective values.

This may prevent the user from making input errors.

(6.7) Seventh Variation

In the embodiment described above, the second acquisition unit 153 is configured to acquire the subjective value, every time the coordinates of a combination of the first mental and physical condition information and the second mental and physical condition information are displayed. However, this configuration is only an example and should not be construed as limiting.

The second acquisition unit 153 does not have to acquire the subjective value every time the coordinates of the combination of the first mental and physical condition information and the second mental and physical condition information are displayed.

For example, when finding the coordinates of the combination of the first mental and physical condition information and the second mental and physical condition information agreeing with a combination of a subjective value with respect to the arousal level and a subjective value with respect to the valence level, the user u1 does not have to enter the combination of the subjective value with respect to the arousal level and the subjective value with respect to the valence level. In that case, unless a combination of a subjective value with respect to the arousal level and a subjective value with respect to the valence level is acquired within a predetermined time, the second acquisition unit 153 decides that the coordinates of the combination of the first mental and physical condition information and the second mental and physical condition information agree with the combination of the subjective value with respect to the arousal level and the subjective value with respect to the valence level. The second acquisition unit 153 stores a corrected estimated value (or an estimated value) included in the first mental and physical condition information as a subjective value with respect to the arousal level in association with the estimated value with respect to the arousal level. The second acquisition unit 153 stores a corrected estimated value (or an estimated value) included in the second mental and physical condition information as a subjective value with respect to the valence level in association with the estimated value with respect to the valence level.

(6.8) Eighth Variation

In the embodiment described above, the condition visualizer 10 is configured to display the mental and physical condition in terms of the two types of levels (namely, the arousal level and the valence level). However, this configuration is only an example and should not be construed as limiting. Alternatively, the condition visualizer 10 may display the mental and physical condition in terms of one of the two types of levels (namely, either the arousal level or the valence level).

(6.9) Ninth Variation

In the embodiment described above, the condition visualizer 10 includes the display unit 13. However, this configuration is only an example and should not be construed as limiting.

The condition visualizer 10 may include no display unit 13. In that case, the output unit 156 has the on-screen image G10 displayed on a monitor screen of a terminal device different from the condition visualizer 10. For example, the output unit 156 may have the on-screen image G10 displayed on a monitor screen of a mobile communications device owned by the user u1. More specifically, the output unit 156 transmits the first mental and physical condition information and the second mental and physical condition information to the mobile communications device via wireless communication, for example, such that the combination of the first mental and physical condition information and the second mental and physical condition information is displayed as coordinates on a two-dimensional matrix. In this case, examples of the mobile communications device include a tablet computer and a smartphone.

Note that even if the condition visualizer 10 includes the display unit 13, the output unit 156 may transmit the first mental and physical condition information and the second mental and physical condition information to the mobile communications device. In that case, the on-screen image G10 will be displayed on both the display unit 13 and the mobile communications device. In other words, the condition visualizer 10 is configured to display the on-screen image G10 on at least one of the display unit 13 or the mobile communications device.

(6.10) Tenth Variation

In the embodiment described above, the condition visualizer 10 is configured to display, before the user u1 takes a nap, the first mental and physical condition information and the second mental and physical condition information that have been estimated before the nap and display, after the user u1 has taken a nap, the first mental and physical condition information and the second mental and physical condition information that have been estimated after the nap. However, this configuration is only an example and should not be construed as limiting.

For example, after the user u1 has taken a nap, the condition visualizer 10 may display not only the first mental and physical condition information and the second mental and physical condition information that have been estimated after the nap but also the first mental and physical condition information and the second mental and physical condition information that have been estimated before the nap.

Furthermore, after the user u1 has taken a nap, the condition visualizer 10 may display not only the first mental and physical condition information and the second mental and physical condition information that have been estimated after the nap but also the first mental and physical condition information and the second mental and physical condition information that have been estimated during the nap. In that case, a line graph showing a trajectory of variation in the first mental and physical condition information and the second mental and physical condition information may be displayed.

(6.11) Eleventh Variation

In the embodiment described above, the condition visualizer 10 is applied to a user u1 who is going to take a nap. However, this is only an exemplary application of the condition visualizer 10 and should not be construed as limiting.

Alternatively, the condition visualizer 10 is also applicable to a situation where biometric information is acquired before and after the user u1 takes an action. For example, the condition visualizer 10 may acquire biometric information which is measured before and after the user u1 does desk work. In that case, the spatial controller 30 controls at least one of the plurality of devices 40 (including the air conditioner 41 and the lighting fixture 42) based on the mental and physical condition information derived from the biometric information that has been acquired before the user u1 does the desk work.

(Other Variations)

Note that the embodiment described above is only an exemplary one of various embodiments of the present disclosure and should not be construed as limiting. Rather, the exemplary embodiment described above may be readily modified in various manners depending on a design choice or any other factor without departing from the scope of the present disclosure. Also, the functions of the condition visualization system 1 may also be implemented as, for example, a condition visualization method, a computer program, or a non-transitory storage medium that stores the program thereon. A condition visualization method for a condition visualization system 1 according to an aspect includes a first acquisition step, an estimation step, a processing step, an output step, a second acquisition step, and a correction information generation step. The first acquisition step includes acquiring a user's u1 biometric information from a measuring unit (e.g., a measuring device 20) that measures the biometric information. The estimation step includes obtaining, based on the biometric information, an estimated value representing the user's u1 mental and physical condition. The processing step includes generating, based on the estimated value, mental and physical condition information about the user's u1 mental and physical condition. The output step includes outputting the mental and physical condition information to a display unit (e.g., a display unit 13) that conducts a display based on the mental and physical condition information. The second acquisition step includes acquiring a subjective value that reflects the user's u1 subjective with respect to his or her own mental and physical condition when the estimated value is obtained. The correction information generation step includes generating, using a plurality of data sets, each including the estimated value and the subjective value, correction information about correction to be made to the estimated value when the mental and physical condition information is generated. The processing step includes generating the mental and physical condition information by making correction to the estimated value based on the correction information. A program according to another aspect is designed to cause a computer system to either function as the condition visualization system 1 or perform the condition visualization method for the condition visualization system 1 described above.

The condition visualization system 1 according to the present disclosure or the agent that performs the condition visualization method for the condition visualization system 1 according to the present disclosure includes a computer system. The computer system includes a processor and a memory as hardware components. The functions of the condition visualization system 1 according to the present disclosure or the agent that performs the condition visualization method for the condition visualization system 1 according to the present disclosure may be performed by making the processor execute a program stored in the memory of the computer system. The program may be stored in advance in the memory of the computer system. Alternatively, the program may also be downloaded through a telecommunications line or be distributed after having been recorded in some non-transitory storage medium such as a memory card, an optical disc, or a hard disk drive, any of which is readable for the computer system. The processor of the computer system may be made up of a single or a plurality of electronic circuits including a semiconductor integrated circuit (IC) or a large-scale integrated circuit (LSI). As used herein, the “integrated circuit” such as an IC or an LSI is called by a different name depending on the degree of integration thereof. Examples of the integrated circuits include a system LSI, a very-large-scale integrated circuit (VLSI), and an ultra-large-scale integrated circuit (ULSI). Optionally, a field-programmable gate array (FPGA) to be programmed after an LSI has been fabricated or a reconfigurable logic device allowing the connections or circuit sections inside of an LSI to be reconfigured may also be adopted as the processor. Those electronic circuits may be either integrated together on a single chip or distributed on multiple chips, whichever is appropriate. Those multiple chips may be aggregated together in a single device or distributed in multiple devices without limitation.

Also, in the embodiment described above, the plurality of functions of the condition visualization system 1 are integrated together in a single housing. However, this is not an essential configuration for the condition visualization system 1. Alternatively, those constituent elements of the condition visualization system 1 may be distributed in multiple different housings. Still alternatively, at least some functions of the condition visualization system 1 (e.g., some functions of the condition visualizer 10) may be implemented as a cloud computing system as well.

(Recapitulation)

As can be seen from the foregoing description, a condition visualization system (1) according to a first aspect includes a first acquisition unit (151), an estimation unit (152), a processing unit (155), an output unit (156), a second acquisition unit (153), and a correction information generation unit (154). The first acquisition unit (151) acquires a user's biometric information from a measuring unit (e.g., a measuring device 20) that measures the biometric information. The estimation unit (152) obtains, based on the biometric information, an estimated value representing the user's (u1) mental and physical condition. The processing unit (155) generates, based on the estimated value, mental and physical condition information about the user's (u1) mental and physical condition. The output unit (156) outputs the mental and physical condition information to a display unit (e.g., display unit 13) that conducts a display based on the mental and physical condition information. The second acquisition unit (153) acquires a subjective value that reflects the user's (u1) subjective with respect to the user's (u1) own mental and physical condition when the estimated value is obtained. The correction information generation unit (154) generates, using a plurality of data sets, each including the estimated value and the subjective value, correction information about correction to be made to the estimated value when the mental and physical condition information is generated. The processing unit (155) generates the mental and physical condition information by making correction to the estimated value based on the correction information.

According to this configuration, the estimated value is corrected based on the correction information that has been generated using the subjective value, thus making the estimated value more approximate to a value that reflects the user's (u1) subjective. This enables notifying the user (u1) of a result of more accurate estimation of his or her own mental and physical condition.

In a condition visualization system (1) according to a second aspect, which may be implemented in conjunction with the first aspect, the estimation unit (152) obtains as a plurality of the estimated values, a first-type estimated value with respect to a first type of condition representing the mental and physical condition and a second-type estimated value with respect to a second type of condition representing the mental and physical condition. The processing unit (155) generates, based on the first-type estimated value, first mental and physical condition information as a piece of the mental and physical condition information and also generates, based on the second-type estimated value, second mental and physical condition information as another piece of the mental and physical condition information. The output unit (156) outputs the first mental and physical condition information and the second mental and physical condition information to the display unit to have a combination of the first mental and physical condition information and the second mental and physical condition information displayed as coordinates on a two-dimensional matrix.

This configuration enables displaying, by a simple method, a mental and physical condition represented by the first mental and physical condition information and the second mental and physical condition information.

In a condition visualization system (1) according to a third aspect, which may be implemented in conjunction with the second aspect, the second acquisition unit (153) acquires, while the display unit is displaying the first mental and physical condition information and the second mental and physical condition information, coordinates specified on the two-dimensional matrix. The second acquisition unit (153) acquires one value, corresponding to the first type, out of two values represented by the coordinates, as the subjective value associated with the first-type estimated value. The second acquisition unit (153) also acquires the other value, corresponding to the second type, out of the two values, as the subjective value associated with the second-type estimated value.

This configuration enables acquiring the subjective value corresponding to the first type and the subjective value corresponding to the second type while the first mental and physical condition information and the second mental and physical condition information are being displayed. In other words, this allows the user to enter, while the first mental and physical condition information and the second mental and physical condition are being displayed, a subjective value corresponding to the first type and a subjective value corresponding to the second type, by reference to the content of the information displayed.

In a condition visualization system (1) according to a fourth aspect, which may be implemented in conjunction with any one of the first to third aspects, the correction information generation unit (154) calculates, using at least a predetermined number of data sets, each having the same estimated value, out of the plurality of data sets, an average value of a plurality of subjective values included in the at least the predetermined number of data sets. The correction information generation unit (154) generates, as the correction information with respect to the estimated value, the average value thus calculated.

This configuration enables generating, as correction information, the average value of the plurality of subjective values.

In a condition visualization system (1) according to a fifth aspect, which may be implemented in conjunction with the fourth aspect, the correction information generation unit (154) calculates, when there are less than the predetermined number of the data sets, each including the estimated value, an average value using subjective values included in a plurality of the data sets including the estimated value and subjective values included in another plurality of the data sets including another estimated value continuous with the estimated value to generate, as the correction information with respect to the estimated value, the average value thus calculated.

This configuration enables generating correction information even when there are less than the predetermined number of data sets.

In a condition visualization system (1) according to a sixth aspect, which may be implemented in conjunction with the fourth or fifth aspect, the correction information includes a corrected estimated value as a corrected value of an associated estimated value. A plurality of the estimated values and a plurality of the corrected estimated values, corresponding to the plurality of the estimated values, are associated with each other. When the plurality of estimated values includes a first estimated value and a second estimated value, of which values are continuous with each other, the first estimated value is smaller than the second estimated value, and a first corrected estimated value, associated with the first estimated value, out of the plurality of corrected estimated values is larger than a second corrected estimated value, associated with the second estimated value, out of the plurality of corrected estimated values, the correction information generation unit (154) generates correction information in the following manner. Specifically, the correction information generation unit (154) calculates an average value of: a plurality of subjective values associated with a third corrected estimated value; a plurality of subjective values associated with the first estimated value; and a plurality of subjective values associated with the second estimated value. The correction information generation unit (154) generates the correction information including the average value as the corrected estimated value associated with the second estimated value. In this case, the third corrected estimated value is a corrected estimated value associated with a third estimated value that is continuous with, and larger than, the second estimated value.

This configuration enables preventing the relationship between magnitudes of the first estimated value and the second estimated value and the relationship between magnitudes of the first corrected estimated value associated with the first estimated value and the second corrected estimated value associated with the second estimated value from being reversed.

In a condition visualization system (1) according to a seventh aspect, which may be implemented in conjunction with any one of the fourth to sixth aspects, the correction information generation unit (154) calculates, when a difference between the estimated value and the subjective value is equal to or greater than a predetermined value in a plurality of the data sets each having the same estimated value, the average value with the subjective value excluded.

According to this configuration, a value with a higher degree of reliability is used as the subjective value, thus enabling calculating a corrected estimated value with a higher degree of reliability.

In a condition visualization system (1) according to an eighth aspect, which may be implemented in conjunction with any one of the first to seventh aspects, the second acquisition unit (153) stores, on acquiring the subjective value, not only the subjective value but also the estimated value representing the mental and physical condition and estimated by the estimation unit (152) in a storage unit (11) in association with each other. The correction information generation unit (154) updates the correction information when an update condition for updating the correction information is satisfied.

This configuration enables generating mental and physical condition information with an even higher degree of reliability by updating the correction information.

A spatial control system (2) according to a ninth aspect includes a spatial controller (30) that performs spatial control based on the mental and physical condition information generated by the condition visualization system (1) according to any one of the first to eighth aspects.

This configuration enables performing spatial control appropriately according to the user's (u1) mental and physical condition.

A condition visualization method according to a tenth aspect includes a first acquisition step, an estimation step, a processing step, an output step, a second acquisition step, and a correction information generation step. The first acquisition step includes acquiring a user's (u1) biometric information from a measuring unit (e.g., a measuring device 20) that measures the biometric information. The estimation step includes obtaining, based on the biometric information, an estimated value representing the user's (u1) mental and physical condition. The processing step includes generating, based on the estimated value, mental and physical condition information about the user's (u1) mental and physical condition. The output step includes outputting the mental and physical condition information to a display unit (e.g., a display unit 13) that conducts a display based on the mental and physical condition information. The second acquisition step includes acquiring a subjective value that reflects the user's (u1) subjective with respect to the user's (u1) own mental and physical condition when the estimated value is obtained. The correction information generation step includes generating, using a plurality of data sets, each including the estimated value and the subjective value, correction information about correction to be made to the estimated value when the mental and physical condition information is generated. The processing step includes generating the mental and physical condition information by making correction to the estimated value based on the correction information.

According to this condition visualization method, the estimated value is corrected based on the correction information that has been generated using the subjective value, thus making the estimated value more approximate to a value that reflects the user's (u1) subjective. This enables notifying the user (u1) of a result of more accurate estimation of his or her own mental and physical condition.

A program according to an eleventh aspect is designed to cause a computer to perform the condition visualization method according to the tenth aspect.

According to this program, the estimated value is corrected based on the correction information that has been generated using the subjective value, thus making the estimated value more approximate to a value that reflects the user's (u1) subjective. This enables notifying the user (u1) of a result of more accurate estimation of his or her own mental and physical condition.

### REFERENCE SIGNS LIST

1 Condition Visualization System
2 Spatial Control System
10 Condition Visualizer
11 Storage Unit
13 Display Unit
20 Measuring Device
30 Spatial Controller
151 First Acquisition Unit
152 Estimation Unit
153 Second Acquisition Unit
154 Correction Information Generation Unit
155 Processing Unit
156 Output Unit
u1 User

1. A condition visualization system comprising:
a first acquisition unit configured to acquire a user's biometric information from a measuring unit configured to measure the biometric information;
an estimation unit configured to obtain, based on the biometric information, an estimated value representing the user's mental and physical condition;
a processing unit configured to generate, based on the estimated value, mental and physical condition information about the user's mental and physical condition;
an output unit configured to output the mental and physical condition information to a display unit configured to conduct a display based on the mental and physical condition information;
a second acquisition unit configured to acquire a subjective value that reflects the user's subjective with respect to the user's own mental and physical condition when the estimated value is obtained; and
a correction information generation unit configured to generate, using a plurality of data sets, each including the estimated value and the subjective value, correction information about correction to be made to the estimated value when the mental and physical condition information is generated,
the processing unit being configured to generate the mental and physical condition information by making correction to the estimated value based on the correction information.

2. The condition visualization system of claim 1, wherein
the estimation unit is configured to obtain, as a plurality of the estimated values, a first-type estimated value with respect to a first type of condition representing the mental and physical condition and a second-type estimated value with respect to a second type of condition representing the mental and physical condition,
the processing unit is configured to generate, based on the first-type estimated value, first mental and physical condition information as a piece of the mental and physical condition information and also generate, based on the second-type estimated value, second mental and physical condition information as another piece of the mental and physical condition information, and
the output unit is configured to output the first mental and physical condition information and the second mental and physical condition information to the display unit to have a combination of the first mental and physical condition information and the second mental and physical condition information displayed as coordinates on a two-dimensional matrix.

3. The condition visualization system of claim 2, wherein
the second acquisition unit is configured to acquire, while the display unit is displaying the first mental and physical condition information and the second mental and physical condition information, coordinates specified on the two-dimensional matrix, also acquire one value, corresponding to the first type, out of two values represented by the coordinates, as the subjective value associated with the first-type estimated value, and further acquire the other value, corresponding to the second type, out of the two values, as the subjective value associated with the second-type estimated value.

4. The condition visualization system of claim 1, wherein
the correction information generation unit is configured to calculate, using at least a predetermined number of data sets, each having the same estimated value, out of the plurality of data sets, an average value of a plurality of subjective values included in the at least the predetermined number of data sets to generate, as the correction information with respect to the estimated value, the average value thus calculated.

5. The condition visualization system of claim 4, wherein
the correction information generation unit is configured to, when there are less than the predetermined number of the data sets, each including the estimated value, calculate an average value using subjective values included in a plurality of the data sets including the estimated value and subjective values included in another plurality of the data sets including another estimated value continuous with the estimated value to generate, as the correction information with respect to the estimated value, the average value thus calculated.

6. The condition visualization system of claim 4, wherein
the correction information includes a corrected estimated value as a corrected value of an associated estimated value,
a plurality of the estimated values and a plurality of the corrected estimated values, corresponding to the plurality of the estimated values, are associated with each other,
when the plurality of estimated values includes a first estimated value and a second estimated value, of which values are continuous with each other, the first estimated value is smaller than the second estimated value, and a first corrected estimated value, associated with the first estimated value, out of the plurality of corrected estimated values is larger than a second corrected estimated value, associated with the second estimated value, out of the plurality of corrected estimated values,
the correction information generation unit is configured to calculate an average value of: a plurality of subjective values associated with a third corrected estimated value, which is a corrected estimated value associated with a third estimated value that is continuous with, and larger than, the second estimated value; a plurality of subjective values associated with the first estimated value; and a plurality of subjective values associated with the second estimated value to generate the correction information including the average value as the corrected estimated value associated with the second estimated value.

7. The condition visualization system of claim 4, wherein
the correction information generation unit is configured to, when a difference between the estimated value and the subjective value is equal to or greater than a predetermined value in a plurality of the data sets each having the same estimated value, calculate the average value with the subjective value excluded.

8. The condition visualization system of claim 1, wherein
the second acquisition unit is configured to store, on acquiring the subjective value, not only the subjective value but also the estimated value representing the mental and physical condition and estimated by the estimation unit in a storage unit in association with each other, and
the correction information generation unit is configured to update the correction information when an update condition for updating the correction information is satisfied.

9. A spatial control system comprising a spatial controller configured to perform spatial control based on the mental and physical condition information generated by the condition visualization system of claim 1.

10. A condition visualization method comprising:
a first acquisition step including acquiring a user's biometric information from a measuring unit configured to measure the biometric information;
an estimation step including obtaining, based on the biometric information, an estimated value representing the user's mental and physical condition;
a processing step including generating, based on the estimated value, mental and physical condition information about the user's mental and physical condition;
an output step including outputting the mental and physical condition information to a display unit configured to conduct a display based on the mental and physical condition information;
a second acquisition step including acquiring a subjective value that reflects the user's subjective with respect to the user's own mental and physical condition when the estimated value is obtained; and
a correction information generation step including generating, using a plurality of data sets, each including the estimated value and the subjective value, correction information about correction to be made to the estimated value when the mental and physical condition information is generated,
the processing step including generating the mental and physical condition information by making correction to the estimated value based on the correction information.

11. A non-transitory computer-readable tangible recording medium storing program designed to cause a computer to perform the condition visualization method of claim 10.
